(19) United States
(12) Patent Application Publication
(10) Pub. No.: US 2025/0281894 A1
(43) Pub. Date: Sep. 11, 2025

(54) MICROARRAYS HAVING A NITROCELLULOSE COATING AND METHODS OF PRODUCTION

(71) Applicants: Schott AG, Mainz (DE); SCHOTT Technical Glass Solutions GmbH, Jena (DE)

(72) Inventors: Johannna Blass, Mainz (DE); Stephanie Mangold, Mainz (DE); Andrea Anton, Mainz (DE); Corinna Schwung, Jena (DE); Dietmar Knoll, Jena (DE)

(73) Assignees: Schott AG, Mainz (DE); SCHOTT Technical Glass Solutions GmbH, Jena (DE)

(21) Appl. No.: 19/073,221

(22) Filed: Mar. 7, 2025

(30) Foreign Application Priority Data
Mar. 8, 2024 (DE) 10 2024 106 741.1

(51) Int. Cl. B01J 19/00

(52) U.S. Cl. CPC B01J 19/0046; B01J 2219/00612; B01J 2219/0063; B01J 2219/00637; B01J 2219/00659

(57) ABSTRACT

A microarray for immobilizing biomolecules includes a glass or glass-ceramic substrate and a nitrocellulose coating disposed at least regionally on a first planar surface of the substrate. The nitrocellulose coating is configured to serve as an immobilization zone for biomolecules. A layer thickness of the nitrocellulose coating is between 10 and 150 nm, the nitrocellulose coating is optically clear, and the nitrocellulose coating has a root mean square (RMS) roughness of at least 0.5 nm.

### CROSS REFERENCE TO RELATED APPLICATIONS

This application claims priority to German Patent Application No. 10 2024 106 741.1 filed on Mar. 8, 2024, which is incorporated in its entirety herein by reference.

### BACKGROUND OF THE INVENTION

### 1. Field of the Invention

The invention relates to microarrays for immobilizing biomolecules and to methods for production thereof. The invention further relates to the use of a microarray both for immobilizing biomolecules and for analyzing biomolecules present in a sample.

### 2. Description of the Related Art

Various microarrays and biochips are known from practice that make it possible for biomolecules present in a sample to be immobilized in a minute space and to be presented for further analysis. In this respect, it should be noted that the term “sample” in the context of this disclosure is to be understood in the broadest sense, and thus includes both body fluids under analysis, for example blood, blood plasma, urine, saliva, etc., and tissues, tissue sections or whole cells isolated therefrom, but also encompasses laboratory-prepared samples, for example cell layers, cell extracts, cell culture media and supernatants, nucleic acid or protein solutions, etc., and such a sample can be immobilized on the surface of the array or chip through the immobilization of the biomolecules present therein and ultimately analyzed.

Reactive coatings for immobilizing biomolecules, for example on the surface of a microarray or biochip, allow the application of a large number of different samples in defined and distinct areas (“spots”) in a minute space (commonly with the aid of robots), with the biomolecules that are to be immobilized as capture probes coming directly into contact with the reactive coating of a microarray or biochip in a spot and, as a result of said contact, being specifically immobilized in the spot for further steps of an analytical method. For example, in a next step, biomolecules matching the capture probes can in turn, as a result of their interaction with the capture probes, be immobilized in the spot, where they can be qualitatively detected, but also commonly quantitatively detected, with the aid of excitable dyes which in turn emit detectable radiation following excitation (so-called fluorophores). In the context of the present disclosure, the term “microarray” expressly encompasses (a) solid substrates having a coating that allow the immobilization of biomolecules, including the application/spotting of primary capture probes, but do not yet have immobilized biomolecules, and (b) solid substrates having a coating on which biomolecules, for example primary capture probes, have already been applied.

DNA microarrays (or DNA chips) commonly consist, for example, of solid substrates, usually glass plates, comprising a layer having different DNA molecules that are chemically fixed as capture probes in discrete spots. By nucleic acid hybridization, the capture probes in a spot interact only with complementary DNA molecules from a complex sample under analysis, and the complementary DNA molecules are therefore specifically immobilized in the spot. The binding between two complementary DNA molecules can then be optically detected and quantified in a subsequent stage of the analytical method, for example by fluorescent labels which are coupled to the sample DNA during the analysis. Thus, microarrays are used, for example, for simultaneously analyzing the expression of a very high number of genes or for diagnosing specific gene mutations.

Moreover, various protein microarrays (or protein chips) are known that can be used in various fields of biological research and diagnostic testing for analyzing complex protein interactions and protein functions. For example, some antibody microarrays use antibodies immobilized in spots in order to detect/measure the presence and concentration of specific proteins in a sample that are specifically recognized by the antibodies. Peptide microarrays comprise peptides which are usually immobilized in spots on a solid substrate and by means of which the interactions between proteins and specific peptide sequences can be analysed. Similarly, proteins purified with protein chip microarrays are applied directly to the chip in order to analyse their interactions with other proteins or biomolecules. Such protein chip microarrays are used, for example, particularly in functional genome analysis and in pharmacology. Another type of protein microarray is reverse-phase protein arrays (RPPAs), which allow the quantitative analysis of protein expression in cell lysates. Protein function microarrays in turn can be used for testing the functions of proteins on a large scale. For example, they can be used to identify enzyme activities or to assess protein-ligand interactions.

In order to be able to ensure biomolecules, in particular nucleic acids and/or proteins present in a sample, are immobilized on a solid substrate suitable for the array, the surface of the substrate must have accordingly suitable binding properties for the particular biomolecules to be bound. In this respect, various reactive surfaces and coatings are known. For example, protein and nucleic acid molecules can react the amino groups of an aminosilanized surface with the carboxyl groups of proteins or nucleic acids and immobilize them on the surface in this way. Chemical activation or functionalization of glass surfaces can be used to provide reactive amino groups and/or epoxy groups for binding nucleic acids and/or proteins on the surface. Polystyrene and polypropylene surfaces can likewise be chemically activated or functionalized for the binding of biomolecules.

Furthermore, microarrays having a nitrocellulose coating for immobilizing biomolecules are known. The binding of biomolecules to nitrocellulose is caused by a combination of weak intermolecular forces, which are likely dominated by hydrophobic interactions and van-der-Waals-forces. This property of nitrocellulose is used in the form of standalone nitrocellulose-membranes in a wide variety of biomedical applications. Nitrocellulose coatings can further have a high density of carbonyl groups resulting from further functionalization of the coatings, which form bonds by reacting with amino groups in nucleic acids and proteins. These properties of nitrocellulose have been used for many years in biotechnology, for example in the preparation of Western blots (protein), Southern blots (DNA) or Northern blots (RNA).

Whereas nitrocellulose membranes have already been used successfully in many diagnostic and analytical applications and methods because of their robustness and practicability, nitrocellulose coatings for microarrays are not without problems. One disadvantage of known nitrocellulose coatings is, for example, a disadvantageous signal-to-noise ratio, i.e. a disadvantageous ratio of the background signal intensity to the signal intensity of an immobilized biomolecule to be detected. The background signal intensity (also known as “background noise”) of a ready-to-use microarray or biochip is made up of the intrinsic fluorescence signal of the coated substrate and the background signal of the coating produced by non-specific binding or immobilization of dye, and can thus overlap with the specific detection signal of the immobilized biomolecule to be detected and adversely affect diagnostic sensitivity. Moreover, this disadvantage can be made worse by the fact that known microarray nitrocellulose coatings commonly require comparatively large spot diameters in order to immobilize a certain amount of sample material. In addition, known nitrocellulose coatings are commonly opaque and hence—depending on layer thickness—light-transmissive only to a limited extent, which further limits their use in optical analytical methods.

Reactive solid substrates used as microarrays are provided, in known fashion, with functional aldehyde, carboxyl, epoxy or amino groups which can form a covalent bond with the biomolecules under analysis in order to attach them permanently to the substrate surface. Although these techniques work quite well for small biomolecules, they tend to be relatively ineffective in the case of larger probe molecules such as antibodies, since the detection thereof may commonly also depend on the orientation and flexibility of the biomolecule under analysis. Such conventional techniques of covalent attachment usually bind only a few biomolecules as capture probes to the surface of the solid substrate in a 2-dimensional layer (2D layer; “monolayer”), and so the binding capacity is low and signal detection is difficult. Since relatively few capture probes are present on the surface of such a 2D array, such systems have moreover a low signal-to-noise ratio for a positive binding reaction between biomolecule and dye. Furthermore, it is often not possible to maintain specific binding of antibodies without sufficient hydration and assistance in or on the coated or functionalized surface of the solid substrate. In the case of 2D microarray formats in which antibodies serve as “capture probes” for the biomolecules under analysis, the service life is thus limited and diagnostic or analytical results may fluctuate depending on the age of the array.

Conventional methods for coating solid substrates for production of microarrays and biochips for the analysis of biological samples encompass all known methods for applying functional layers by means of dipping, spraying and/or spin coating technology. Various coating methods are also known in relation to the coating of solid substrates with a nitrocellulose coating for use as microarrays. However, the reliable application of uniformly reproducible layers commonly requires the use of various solvents and detergents and also a sequence of complex process steps.

### SUMMARY OF THE INVENTION

It is therefore an object of the present invention to design and develop a microarray for immobilizing biomolecules of the kind mentioned at the start such that the microarray has a high specific binding capacity at a small spot diameter, an advantageous signal-to-noise ratio, and a high sensitivity in the case of a sample under analysis having low concentrations of the biomolecules to be immobilized. In this respect, it should be noted that the term “sample” in the context of this disclosure is to be understood in the broadest sense, and thus includes both body fluids under analysis, for example blood, blood plasma, urine, saliva, etc., and tissues, tissue sections or whole cells isolated therefrom, but also encompasses laboratory-prepared samples, for example cell layers, cell extracts, cell culture media and supernatants, nucleic acid or protein solutions, etc., and biomolecules present in such a sample can be immobilized by direct or indirect binding to the immobilization layer of the microarray and ultimately analysed.

In some embodiments provided according to the invention, a microarray for immobilizing biomolecules includes a glass or glass-ceramic substrate and a nitrocellulose coating disposed at least regionally on a first planar surface of the substrate. The nitrocellulose coating is configured to serve as an immobilization zone for biomolecules. A layer thickness of the nitrocellulose coating is between 10 and 150 nm, the nitrocellulose coating is optically clear, and the nitrocellulose coating has a root mean square (RMS) roughness of at least 0.5 nm.

In some embodiments provided according to the invention, a method for producing a microarray includes: providing a coating solution, the coating solution including between 0.2% and 3.0% by weight of highly pure nitrocellulose in an organic solvent, between 0.5% and 10.0% by weight of (3-glycidyloxypropyl) trimethoxysilane (GPTS), and between 0.3% and 20.0% by weight of water; applying the coating solution to a glass or glass-ceramic substrate by dipping the substrate into the coating solution and then withdrawing it from the coating solution; and depositing a nitrocellulose coating from the coating solution on a first planar surface of the substrate by drying, the nitrocellulose coating having a layer thickness of between 10 and 150 nm. The deposited nitrocellulose coating is optically clear and has a root mean square (RMS) roughness of at least 0.5 nm.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIGS. 1A-1C show—according to Example 1—atomic force microscopy images showing the surface quality of different nitrocellulose coatings defining the immobilization zone of various microarrays; FIG. 1A shows surface quality of the nitrocellulose coating of the microarray according to the invention (SCHOTT NC-Array); FIG. 1B shows surface quality of a comparably thin nitrocellulose coating of a known microarray (PATH® Protein Microarray Slide); and FIG. 1C shows surface quality of a nitrocellulose coating having a layer thickness in the micrometre range of a known microarray (ONCYTE® SuperNOVA);

FIG. 2 shows—according to Example 1—the relevant roughness parameters of the nitrocellulose coating of the microarray according to the invention of FIG. 1A compared to those of the nitrocellulose coating of the known microarray of FIG. 1B: root mean square (RMS) roughness; roughness in terms of the mean of the absolute deviations (Sa); and roughness in terms of the peak-to-valley height (Sz);

FIG. 3 shows—according to Example 2—the transmittance spectrum of a cleaned glass substrate (Reference); of the corresponding glass substrate provided with the nitrocellulose coating of the microarray according to the invention (SCHOTT NC-Array), of a known microarray having a comparably thin nitrocellulose coating (PATH® Protein Microarray Slide) and of a known microarray having a nitrocellulose coating with a layer thickness in the micrometre range (ONCYTE® SuperNOVA);

FIG. 4 shows—according to Example 3—the results of the measurements of background intensity of microarrays provided with nitrocellulose coatings versus nitrocellulose layer thickness;

FIG. 5 shows—according to Example 4—the results of measurements of background intensity of the microarray according to the invention (SCHOTT NC-Array) compared to three known microarray formats (SCHOTT Slide E and ONCYTE® and PATH® from Grace Biolabs);

FIGS. 6A and 6B show—according to Example 5—the spot morphology of a sample applied to the nitrocellulose coating of the microarray according to the invention compared to the spot morphology of a corresponding sample on a thick nitrocellulose coating; and

FIGS. 7A and 7B show—according to Example 6—the extension of the dynamic range of the microarray according to the invention (SCHOTT NC-Array) compared to a 2D epoxy microarray (SCHOTT Slide E).

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

In some embodiments, the present invention achieves the above-mentioned object with a microarray for immobilizing biomolecules, comprising (a) a glass or glass-ceramic substrate, and (b) a nitrocellulose coating, the nitrocellulose coating being disposed at least regionally on a first planar surface of the substrate, and the nitrocellulose coating serving as an immobilization zone for biomolecules; characterized in that the layer thickness of the nitrocellulose coating is between 30 and 150 nm, and the nitrocellulose coating is optically clear and has a root mean square (RMS) roughness of at least 0.5 nm.

A particularly advantageous property of the nitrocellulose coating of the aforementioned microarray is that it still has a rough surface quality despite a low layer thickness of between 10 and 150 nm. Nitrocellulose coatings are known as mentioned at the start. Especially nitrocellulose coatings and membranes with layer thicknesses of several micrometres (μm) are commonly three-dimensional and porous and have a high degree of roughness as a result (see Sauer U. Analytical Protein Microarrays: Advancements Towards Clinical Applications. Sensors (Basel). 2017 Jan. 29; 17(2):256. doi: 10.3390/s17020256. PMID: 28146048; PMCID: PMC5335935). Although the three-dimensional and porous nature of such known nitrocellulose coatings increases the binding capacity, it commonly also leads to disadvantageously high background fluorescence of a glass or glass-ceramic substrate coated in such a way, meaning that only a low signal-to-noise ratio is achieved. A low signal-to-noise ratio limits, inter alia, the dynamic range of a microarray. However, a broad dynamic range is important for ensuring accurate and comprehensive detection and analysis of biomolecules which are present in a sample and differ in concentration or expression level. If the dynamic range is too narrow, important information may be lost, especially when analyzing biomolecules differing greatly in concentration or expression level.

Moreover, the surface quality of a nitrocellulose coating influences the morphology of the samples applied on a microarray in individual spots. In the case of known nitrocellulose coatings and membranes with layer thicknesses of several micrometres (μm), spot diameters of more than 200 μm, often up to 400 μm, are commonly necessary in order to be able to apply a sufficient sample volume per spot.

The inventors initially recognized that the signal-to-noise ratio of a nitrocellulose coating can be increased in an astonishingly simple manner by reducing the layer thickness while simultaneously forming a microstructure on the surface of the nitrocellulose coating. Since the lower layer thickness reduces the non-specific binding capacity compared to a thicker 3D nitrocellulose layer, the stated surface microstructure of the thin nitrocellulose coating can—compared to a monolayer of reactive groups—provide a larger number of reactive groups per unit of area, thus achieving an advantageous specific binding capacity. Moreover, such a configuration extends the dynamic range of a microarray according to the invention compared to a microarray having a 2D monolayer of reactive epoxy groups.

Optionally, the layer thickness of the nitrocellulose coating of the microarray according to the invention is between 10 and 100 nm, in particular between 20 and 100 nm, in particular between 30 and 100 nm, in particular between 30 and 90 nm, in particular between 35 and 85 nm, in particular between 40 and 80 nm, in particular between 45 and 75 nm, or is approximately 45 nm, 50 nm, 55 nm, 60 nm, 65 nm, 70 nm, 75 nm, 80 nm or 85 nm.

Microarrays having nitrocellulose coatings with a layer thickness of less than 200 nm are likewise known. However, the thin nitrocellulose coatings of such known microarrays are substantially smooth.

Despite a low layer thickness, the nitrocellulose coating of the microarray according to the invention has, as already mentioned, a root mean square (RMS) roughness of at least 0.5 nm, optionally between 0.5 and 2 nm, in particular between 0.75 and 2 nm, in particular between 1 and 2 nm, in particular between 1.25 and 2 nm, in particular between 1.25 and 1.75 nm, in particular between 1.45 and 1.65 nm, or of approximately 1.3 nm, 1.4 nm, 1.5 nm, 1.6 nm or 1.7 nm.

The root mean square value (“RMS value” or “Sq”) is frequently used in relation to the quality of surfaces and in measurement techniques to describe the roughness of a surface. The RMS value indicates the extent of the deviations on a surface from an ideal smooth surface.

The formula for calculating the RMS value for the roughness of a surface is:

$$Sq = \sqrt{\frac{1}{a} \int {\int }_{a} {( Z ( x , y ) )}^{2} dxdy}$$

Herein Z(x,y) denotes the height Z of the surface over a reference surface at the location with coordinates (x,y). A low RMS value indicates that the surface is relatively smooth and homogeneous, whereas a higher RMS value indicates a textured or rougher surface with larger deviations.

The average roughness of a surface over its entire three-dimensional structure can be indicated by the mean of the absolute deviations (Sₐ).

$${S}_{a} = \frac{1}{a} \int {\int }_{a} \vert Z ( x , y ) \vert dxdy$$

The rough nitrocellulose coating of the microarray according to the invention has a roughness in terms of the mean of the absolute deviations (Sₐ) of optionally between 0.3 and 1 nm, in particular between 0.3 and 0.9 nm, in particular between 0.3 and 0.7 nm, in particular between 0.4 and 0.7 nm, in particular between 0.5 and 0.7 nm, in particular between 0.5 and 0.6 nm, or of approximately 0.4 nm, 0.45 nm, 0.5 nm, 0.55 nm, 0.6 nm, 0.65 nm or 0.7 nm.

Another parameter for characterizing the roughness of a surface is the measurable peak-to-valley height (S_{z}), i.e. the maximum height difference between the height of the highest peak (Sₚ) and the depth of the lowest valley (Sᵥ) for a rough surface.

$${S}_{z} = {S}_{p} - {S}_{v}$$

The rough nitrocellulose coating of the microarray according to the invention has a roughness in terms of the peak-to-valley height (S_{z}) of optionally between 150 and 300 nm, in particular between 150 and 275 nm, in particular between 150 and 250 nm, in particular between 175 and 250 nm, in particular between 190 and 230 nm, in particular between 210 and 230 nm, or of approximately 190 nm, 200 nm, 215 nm, 220 nm, 225 nm, 230 nm or 240 nm.

As described in Example 1, the surface quality of the nitrocellulose coating of the microarray according to the invention distinctly differs with regard to the microstructure thereof from the surface quality of known microarrays having nitrocellulose coatings of comparably low layer thickness. The above-mentioned roughness of the surface of the nitrocellulose coating of a microarray according to the invention is the objectively measurable expression of this microstructure.

The nitrocellulose coating of the microarray according to the invention is optically clear. Here, “optically clear” in relation to the nitrocellulose coating means that the transmittance of the microarray substantially corresponds to that of the glass or glass-ceramic substrate without a nitrocellulose coating and the coating itself has a transmittance of more than 90% in the wavelength range between 400 and 700 nm. As described in Example 2, the microarray according to the invention has a transmittance in the wavelength range between 300 and 800 nm of at least 85%, optionally between 85% and 99%, in particular between 90% and 99%, in particular between 90% and 95%, or the transmittance is approximately 85%, 87.5%, 90%, 92.5%, 95% or 97.5%.

A glass or glass-ceramic substrate of the microarray according to the invention itself (i.e. without the nitrocellulose coating) likewise has a transmittance in the wavelength range between 300 and 800 nm of at least 85%, optionally between 85% and 99%, in particular between 90% and 99%, in particular between 90% and 95%, or the transmittance is approximately 85%, 87.5%, 90%, 92.5%, 95% or 97.5%.

In some embodiments, the microarray according to the invention is optionally designed such that
the layer thickness of the rough nitrocellulose coating is between 45 and 75 nm,
the nitrocellulose coating has a root mean square (RMS) roughness of between 1.45 and 1.65 nm,
the rough nitrocellulose coating has a roughness in terms of the mean of the absolute deviations (Sa) of between 0.5 and 0.6,
the rough nitrocellulose coating has a roughness in terms of the peak-to-valley height (Sz) of between 210 and 230, and
the microarray has a transmittance in the wavelength range between 300 and 800 nm of between 90% and 95%.

With respect to a glass substrate to be used in the aforementioned microarrays, it is conceivable that said substrate is selected from: soda-lime glasses, borosilicate glasses, quartz glasses and/or alkali metal-free aluminoborosilicate glasses.

Optionally, the glass substrate to be used in the microarray according to the invention may have the following composition components in line with a lithium aluminium silicate glass (in % by weight):

| | |
|---|---|
| SiO₂ | 55-69 |
| Al₂O₃ | 18-25 |
| Li₂O | 3-5 |
| Na₂O + K₂O | 0-30 |
| MgO + CaO + SrO + BaO | 0-5 |
| ZnO | 0-4 |
| TiO₂ | 0-5 |
| ZrO₂ | 0-5 |
| TiO2 + ZrO₂ + SnO₂ | 2-6 |
| P₂O₅ | 0-8 |
| F | 0-1 |
| B₂O₃ | 0-2 |

Again optionally, the glass substrate to be used in the microarray according to the invention may have the following components (in % by weight):

| | |
|---|---|
| SiO₂ | 57-66 |
| Al₂O₃ | 18-23 |
| Li₂O | 3-5 |
| Na₂O + K₂O | 3-25 |
| MgO + CaO + SrO + BaO | 1-4 |
| ZnO | 0-4 |
| TiO₂ | 0-4 |
| ZrO₂ | 0-5 |
| TiO2 + ZrO₂ + SnO₂ | 2-6 |
| P₂O₅ | 0-7 |
| F | 0-1 |
| B₂O₃ | 0-2 |

Again optionally, the glass substrate to be used in the microarray according to the invention may have the following components (in % by weight):

| | |
|---|---|
| SiO₂ | 57-63 |
| Al₂O₃ | 18-22 |
| Li₂O | 3.5-5 |
| Na₂O + K₂O | 5-20 |
| MgO + CaO + SrO + BaO | 0-5 |
| ZnO | 0-3 |
| TiO₂ | 0-3 |
| ZrO₂ | 0-5 |
| TiO2 + ZrO₂ + SnO₂ | 2-5 |
| P₂O₅ | 0-5 |
| F | 0-1 |
| B₂O₃ | 0-2 |

Again optionally, the glass substrate to be used in the microarray according to the invention may have the following components in line with a soda-lime silicate glass (in % by weight):

| | |
|---|---|
| SiO₂ | 40-81 |
| Al₂O₃ | 0-6 |
| B₂O₃ | 0-5 |
| Li₂O + Na₂O + K₂O | 5-30 |
| MgO + CaO + SrO + BaO + ZnO | 5-30 |
| TiO₂ + ZrO₂ | 0-7 |
| P₂O₅ | 0-2 |

Again optionally, the glass substrate to be used in the microarray according to the invention may have the following components (in % by weight):

| | |
|---|---|
| SiO₂ | 50-81 |
| Al₂O₃ | 0-5 |
| B₂O₃ | 0-5 |
| Li₂O + Na₂O + K₂O | 5-28 |
| MgO + CaO + SrO + BaO + ZnO | 5-25 |
| TiO₂ + ZrO₂ | 0-6 |
| P₂O₅ | 0-2 |

Again optionally, the glass substrate to be used in the microarray according to the invention may have the following components (in % by weight):

| | |
|---|---|
| SiO₂ | 50-76 |
| Al₂O₃ | 0-5 |
| B₂O₃ | 0-5 |
| Li₂O + Na₂O + K₂O | 5-25 |
| MgO + CaO + SrO + BaO + ZnO | 5-20 |
| TiO₂ + ZrO₂ | 0-5 |
| P₂O₅ | 0-2 |

Again optionally, the glass substrate to be used in the microarray according to the invention may have the following components in line with a borosilicate glass (in % by weight):

| | |
|---|---|
| SiO₂ | 60-85 |
| Al₂O₃ | 0-10 |
| B₂O₃ | 5-20 |
| Li₂O + Na₂O + K₂O | 2-16 |
| MgO + CaO + SrO + BaO + ZnO | 0-15 |
| TiO₂ + ZrO₂ | 0-5 |
| P₂O₅ | 0-2 |

Again optionally, the glass substrate to be used in the microarray according to the invention may have the following components (in % by weight):

| | |
|---|---|
| SiO₂ | 63-84 |
| Al₂O₃ | 0-8 |
| B₂O₃ | 5-18 |
| Li₂O + Na₂O + K₂O | 3-14 |
| MgO + CaO + SrO + BaO + ZnO | 0-12 |
| TiO₂ + ZrO₂ | 0-4 |
| P₂O₅ | 0-2 |

Again optionally, the glass substrate to be used in the microarray according to the invention may have the following components (in % by weight):

| | |
|---|---|
| SiO₂ | 63-83 |
| Al₂O₃ | 0-7 |
| B₂O₃ | 5-18 |
| Li₂O + Na₂O + K₂O | 4-14 |
| MgO + CaO + SrO + BaO + ZnO | 0-10 |
| TiO₂ + ZrO₂ | 0-3 |
| P₂O₅ | 0-2 |

Again optionally, the glass substrate to be used in the microarray according to the invention may have the following components (in % by weight):

| | |
|---|---|
| SiO₂ | 60-70 |
| Al₂O₃ | 1-10 |
| B₂O₃ | 1-10 |
| K₂O | 1-10 |
| Na₂O | 1-10 |
| ZnO | 1-10 |
| TiO₂ | 1-10 |

Again optionally, the glass substrate to be used in the microarray according to the invention may have the following components in line with an alkali metal aluminium silicate glass (in % by weight):

| | |
|---|---|
| SiO₂ | 40-75 |
| Al₂O₃ | 10-30 |
| B₂O₃ | 0-20 |
| Li₂O + Na₂O + K₂O | 4-30 |
| MgO + CaO + SrO + BaO + ZnO | 0-15 |
| TiO₂ + ZrO₂ | 0-15 |
| P₂O₅ | 0-10 |

Again optionally, the glass substrate to be used in the microarray according to the invention may have the following components in accordance with a low-alkali metal aluminium silicate glass (in % by weight):

| | |
|---|---|
| SiO₂ | 50-70 |
| Al₂O₃ | 10-27 |
| B₂O₃ | 0-18 |
| Li₂O + Na₂O + K₂O | 5-28 |
| MgO + CaO + SrO + BaO + ZnO | 0-13 |
| TiO₂ + ZrO₂ | 0-13 |
| P₂O₅ | 0-9 |

Again optionally, the glass substrate to be used in the microarray according to the invention may have the following components (in % by weight):

| | |
|---|---|
| SiO₂ | 55-68 |
| Al₂O₃ | 10-27 |
| B₂O₃ | 0-15 |
| Li₂O + Na₂O + K₂O | 4-27 |
| MgO + CaO + SrO + BaO + ZnO | 0-12 |
| TiO₂ + ZrO₂ | 0-10 |
| P₂O₅ | 0-8 |

Again optionally, the glass substrate to be used in the microarray according to the invention may have the following components (in % by weight):

| | |
|---|---|
| SiO₂ | 50-75 |
| Al₂O₃ | 7-25 |
| B₂O₃ | 0-20 |
| Li₂O + Na₂O + K₂O | 0-4 |
| MgO + CaO + SrO + BaO + ZnO | 5-25 |
| TiO₂ + ZrO₂ | 0-10 |
| P₂O₅ | 0-5 |

Again optionally, the glass substrate to be used in the microarray according to the invention may have the following components (in % by weight):

| | |
|---|---|
| SiO₂ | 52-73 |
| Al₂O₃ | 7-23 |
| B₂O₃ | 0-18 |
| Li₂O + Na₂O + K₂O | 0-4 |
| MgO + CaO + SrO + BaO + ZnO | 5-23 |
| TiO₂ + ZrO₂ | 0-10 |
| P₂O₅ | 0-5 |

Again optionally, the glass substrate to be used in the microarray according to the invention may have the following components (in % by weight):

| | |
|---|---|
| SiO₂ | 53-71 |
| Al₂O₃ | 7-22 |
| B₂O₃ | 0-18 |
| Li₂O + Na₂O + K₂O | 0-4 |
| MgO + CaO + SrO + BaO + ZnO | 5-22 |
| TiO₂ + ZrO₂ | 0-8 |
| P₂O₅ | 0-5 |

It will be understood that the respective glass constituents of the glass compositions listed must add up to 100% by weight. Nevertheless, the glasses to be used in the invention, in particular the glasses described above, may in turn be modified. For example, the color of the respective glass may be altered by adding color oxides.

In some configurations, the glass substrates according to the invention are produced using particularly pure raw materials in order to minimize the fluorescence under illumination with UV radiation and/or visible light radiation. Especially the use of raw materials having a very low iron content has been found to be advantageous for this purpose. The glasses produced in this way thus may advantageously contain particularly few impurities, in particular little iron.

A person skilled in the art will know how to ascertain the background intensity in relation to the signal intensity to be determined in a fluorescence-based measurement system. For example, this may be achieved by using a microplate reader from TECAN, in which both the background intensity and the signal intensity to be determined of the microarray according to the invention is measured by an optical system. In said system, the immobilization zone is initially irradiated by a light source with a wavelength of 532 nm in excitation mode. This results in fluorescence that is emitted by the surface. The relevant emission wavelength is selected by an optical filter, for example with a wavelength of 575 nm. The intensity of the emitted light can then be detected by a detector. With such fluorescence measurements, it may be necessary to set a signal gain, which can of course greatly influence the fluorescence measurements. In this respect, a person skilled in the art knows that it is precisely the defining of this parameter in any measurement system that is important in order to be able to detect a low background intensity in relation to the signal intensity to be determined.

Optionally, the glass or glass-ceramic substrate of the microarray emits only a very low fluorescence signal itself after excitation with a wavelength of 532 nm, such that the background signal intensity of the microarray, which is due to the intrinsic fluorescence signal of the coated substrate and the background signal produced by non-specific binding or immobilization of dyes on the nitrocellulose coating, usually remains well below 100 relative fluorescence units (rfu), in particular between 10 and 50 rfu, after excitation with a wavelength of 532 nm and a gain of 140.

In particular, the intrinsic fluorescence of the microarray is less than 50, optionally between 15 and 40, relative fluorescence units at an excitation wavelength of 532 nm and a gain of 140.

Example 3 shows that the intrinsic fluorescence of a microarray increases as the nitrocellulose layer thickness increases, and that the intrinsic fluorescence of a microarray according to the invention having a nitrocellulose coating with a layer thickness of less than 150 nm is advantageously low and differs especially advantageously from the intrinsic fluorescence of a microarray having a nitrocellulose coating with a layer thickness of several micrometres.

Moreover, the nitrocellulose coating of the microarray according to the invention is distinguished by its stable bond with the glass or glass-ceramic substrate, such that the microarray is stable when stored at between 4° C. and 40° C., in particular between 10° C. and 30° C., in particular between 18° C. and 25° C., and long storage stability of the microarray can be ensured even at room temperature. Optionally, the microarray remains stable when stored at between 4° C. and 40° C., in particular between 10° C. and 30° C., in particular between 18° C. and 25° C., for more than 2 months, in particular for more than 4 months, more than 6 months, more than 12 months, more than 18 months or more than 24 months.

In some embodiments of the microarray according to the invention, the first planar surface of the substrate, on which the nitrocellulose coating is disposed at least regionally, is not functionalized. In the context of this application, this means that the surface has not been chemically modified in order to specifically improve the binding properties with respect to the nitrocellulose coating. Nevertheless, the microarrays exhibit the characteristic stability.

In some embodiments of the microarray according to the invention, the first planar surface of the substrate, on which the nitrocellulose coating is disposed at least regionally, is functionalized. The functionalized first planar surface of the substrate comprises as desired:
functional ester groups, optionally N-hydroxysuccinimide ester groups, glycidyl ester groups or isocyanate ester groups;
functional ether groups, optionally glycidyl ether groups;
functional epoxy groups;
functional aldehyde groups;
functional free carboxyl groups; and/or
functional free amino groups; and combinations thereof.

Optionally, the first planar surface of the substrate is functionalized by an adhesion promoter layer which comprises the
functional ester groups, optionally N-hydroxysuccinimide ester groups, glycidyl ester groups or isocyanate ester groups;
functional ether groups, optionally glycidyl ether groups;
functional epoxy groups;
functional aldehyde groups;
functional free carboxyl groups; and/or
functional free amino groups; and combinations thereof.

The term “biomolecule” in the context of this disclosure encompasses both high-molecular-weight macromolecules (such as proteins, nucleic acids, polysaccharides or lipids) and low-molecular-weight compounds which, for example, are the building blocks of the high-molecular-weight macromolecules (such as amino acids, nucleotides, sugars or fatty acids).

Typically, the biomolecules to be immobilized are: nucleic acids, in particular deoxyribonucleic acids (DNA) or ribonucleic acids (RNA); peptides; or proteins, in particular enzymes or antibodies. Nucleic acids to be analysed may be in particular DNA or RNA oligonucleotides.

In the context of this disclosure, biomolecules to be immobilized are usually present in an aqueous sample.

As already described, the microstructure due to the roughness of the surface of the nitrocellulose coating leads, surprisingly, to an advantageously increased binding capacity of the membrane for biomolecules to be immobilized, compared to other nitrocellulose coatings with a comparably low layer thickness, and to improved spot morphology.

In some embodiments, the immobilization zone defined by the nitrocellulose coating may encompass the entire planar surface of the substrate. In such embodiments, the entire surface is thus able to immobilize biomolecules. As is known however, one advantage of microarrays is the application of a large number of different samples in defined and distinct areas (“spots”) in a minute space (commonly with the aid of robots). In some embodiments, a large number of individual, distinct spots may thus be arranged in the immobilization zone. In this respect, the surface quality of the nitrocellulose coating of the microarray according to the invention and the resultant spot morphology is likewise advantageous.

The advantageous surface quality of the nitrocellulose coating means that relatively large volumes of aqueous sample solutions can be applied in spots of small diameter, for example a diameter of between 130 and 170 μm, to the surface provided with the nitrocellulose coating, which in turn ensures an advantageously high concentration of biomolecules per spot.

In particular, a spotting volume of 500 picolitres (pl) applied in the immobilization zone has a spot diameter of between 130 and 170 μm.

The combination of such a large number of biomolecules per spot and the advantageously high binding capacity of the nitrocellulose coating leads to a high signal intensity of biomolecules to be analyzed in the subsequent analytical methods, which significantly facilitates evaluation thereof.

For the labelling and detection of immobilized biomolecules, use is usually made of excitable dyes, so-called fluorophores, which emit detectable radiation following excitation. Examples of suitable fluorophores include:

| | Absorbing | Emitting | Visible |
|---|---|---|---|
| Name | wavelength | wavelength | colour |
| Hydroxycoumarin | 325 | 386 | blue |
| Methoxycoumarin | 360 | 410 | blue |
| Alexa Fluor | 345 | 442 | blue |
| Aminocoumarin | 350 | 445 | blue |
| Cy2 | 490 | 510 | dark green |
| FAM | 495 | 516 | dark green |
| Alexa Fluor 488 | 494 | 517 | light green |
| Fluorescein FITC | 495 | 518 | light green |
| Alexa Fluor 430 | 430 | 545 | light green |
| Alexa Fluor 532 | 530 | 555 | light green |
| HEX | 535 | 556 | light green |
| Cy3 | 550 | 575 | yellow |
| TRITC | 547 | 572 | yellow |
| Alexa Fluor 546 | 556 | 573 | yellow |
| Alexa Fluor 555 | 556 | 573 | yellow |
| R-phycoerythrin (PE) | 480; 565 | 578 | yellow |
| Rhodamine Red-X | 560 | 580 | orange |
| Tamara | 565 | 580 | red |
| Cy3.5 | 581 | 596 | red |
| ROX | 575 | 602 | red |
| Alexa Fluor 568 | 578 | 603 | red |
| Red 613 | 480; 565 | 613 | red |
| Texas Red | 615 | 615 | red |
| Alexa Fluor 594 | 590 | 617 | red |
| Alexa Fluor 633 | 621 | 639 | red |
| Allophycocyanin | 650 | 660 | red |
| Alexa Fluor 633 | 650 | 668 | red |
| Cy5 | 650 | 670 | red |
| Alexa Fluor 660 | 663 | 690 | red |
| Cy5.5 | 675 | 694 | red |
| TruRed | 490; 675 | 695 | red |
| Alexa Fluor 680 | 679 | 702 | red |
| Cy7 | 743 | 770 | red |

Particularly suitable for labelling DNA or RNA molecules are cyanine fluorophores, including the known fluorophores Cy2, Cy3, Cy3B, Cy 3.5, Cy5, Cy5.5 or Cy7. Optionally, a microarray according to the invention has only a low background intensity. Said low background intensity ensures that signals emitted by the fluorophores in the wavelength range from approximately 500 nm to 575 nm can be detected substantially without any interfering background signals from the substrate itself. Advantageously, the sensitivity of a microarray or biochip produced on the basis of the solid substrate according to the invention is therefore high.

The low intrinsic fluorescence of the microarray according to the invention, and the advantageous binding capacity coupled with low non-specific binding and, at the same time, improved spot morphology on the nitrocellulose coating in the immobilization zone of the microarray according to the invention, leads to a high signal-to-noise ratio in the case of samples having a low concentration of biomolecules to be immobilized.

For example, the microarray according to the invention allows the reliable detection and analysis of biomolecules even at antibody concentrations of 0.01 to 0.1 mg/ml.

The present invention also provides a method for producing the aforementioned microarray and provides a microarray produced in this way.

In some embodiments of the invention, the above-described microarrays provided with a nitrocellulose coating may be produced in an astonishingly simple and cost-effective manner by a method according to the invention.

In some embodiments, the present invention achieves the above-mentioned object with a method for producing a microarray according to the invention, the method at least comprising: (a) providing a coating solution; (b) applying the coating solution to a glass or glass-ceramic substrate by dipping the substrate into the coating solution and then withdrawing it from the coating solution; (c) depositing a nitrocellulose coating from the coating solution on a first planar surface of the substrate by drying, the drying optionally comprising evaporating an organic solvent of the coating solution and the nitrocellulose coating having a layer thickness of between 10 and 150 nm, characterized in that the coating solution comprises: between 0.2% and 5.0% by weight of highly pure nitrocellulose in an organic solvent; between 0.5% and 5.0% by weight of (3-glycidyloxypropyl)trimethoxysilane (GPTS); and between 0.3% and 20.0% by weight of water and the deposited nitrocellulose coating is optically clear and has a root mean square (RMS) roughness of at least 0.5 nm.

Optionally, the coating solution additionally comprises: between 0.01% and 3.0% by weight of biotin; or between 0.01% and 3.0% by weight of streptavidin.

The use of biotin or streptavidin in the coating solution may be advantageous, since the combination of biotin and streptavidin is a robust way for reproducibly establishing a stable and selective bond—selective with respect to the binding partners—between the microarray and the biomolecules under study of a sample of interest. Thus, in the case of biotin in the coating solution, the biotin then present on the microarray will bind to streptavidin in a studied sample. Analogously, streptavidin present on the microarray would form a stable bond with biotin present in a sample. A bond between sample and microarray would only form if the complementary partners biotin and streptavidin meet, and so the bonds formed by the coating are selective with regard to the possible binding partners. In effect, adding biotin or streptavidin in the coating solution would thus expand the available coupling systems for fixing biomolecules.

The coating solution optionally comprises between 0.01% and 2.5% by weight of streptavidin or biotin, optionally between 0.05% and 2% by weight of streptavidin or biotin, optionally between 0.1% and 1% by weight of streptavidin.

Optionally, the coating solution comprises:
between 0.5% and 1.5% by weight, optionally between 0.6% and 1.4% by weight or between 0.7% and 1.3% by weight or between 0.8% and 1.2% by weight or between 0.9% and 1.1% by weight or 1% by weight of highly pure nitrocellulose in an organic solvent;
between 2.0% and 4.0% by weight, optionally between 2.1% and 3.9% by weight or between 2.2% and 3.8% by weight or between 2.3% and 3.7% by weight or between 2.4% and 3.6% by weight or between 2.5% and 3.5% by weight or between 2.6% and 3.4% by weight or between 2.7% and 3.3% by weight or between 2.8% and 3.2% by weight or between 2.9% and 3.1% by weight or 3% by weight of GPTS; and
between 0.3% and 15.0% by weight, optionally between 0.5% and 10.0% by weight or between 1.0% and 9.0% by weight or between 2.0% and 8.0% by weight or between 3.0% and 7.0% by weight or between 4.0% and 6.0% by weight or between 4.1% and 5.9% by weight or between 4.2% and 5.8% by weight or between 4.3% and 5.7% by weight or between 4.4% and 5.6% by weight or between 4.5% and 5.5% by weight or between 4.6% and 5.4% by weight or between 4.7% and 5.3% by weight or between 4.8% and 5.2% by weight or between 4.9% and 5.1% by weight or 5% by weight of water.

In some embodiments of the method according to the invention, the coating solution comprises:
between 0.8% and 1.2% by weight of highly pure nitrocellulose in an organic solvent;
between 2.8% and 3.2% by weight of GPTS; and
between 4.8% and 5.2% by weight of water.

In some embodiments of the method according to the invention, the coating solution comprises:
1% by weight of highly pure nitrocellulose in an organic solvent;
3% by weight of GPTS; and
5% by weight of water.

Moreover, the drawing speed for dipping is between 5 and 15 cm per minute, in particular between 8 and 12 cm per minute, in particular 10 cm per minute, at a temperature of the coating solution of between 18° C. and 25° C.

Surprisingly, the aforementioned relative quantitative ratios in the ranges:
of between 0.2% and 3.0% by weight of highly pure nitrocellulose in an organic solvent;
of between 0.5% and 5.0% by weight of (3-glycidyloxypropyl) trimethoxysilane (GPTS); and
of between 0.3% and 20.0% by weight of water, are necessary for applying the nitrocellulose coating having the properties described for the microarray according to the invention to at least a first planar surface of the glass or glass-ceramic substrate.

Optionally, the drying to deposit the nitrocellulose from the coating solution on the surface takes place over a period of at least 5 minutes at a temperature between 18° C. and 70° C., optionally over a period of 20 minutes at a temperature of 60° C.

In some embodiments, the organic solvent may be amyl acetate, isopropanol, 1-propanol, tetrahydrofuran or dimethyl sulfoxide. Especially the aprotic non-polar organic solvent amyl acetate has been found to be suitable for dissolving the highly pure nitrocellulose.

The method according to the invention allows the production of large quantities of the microarrays according to the invention having particularly high stability in an astoundingly simple manner.

Furthermore, the present invention achieves the above-mentioned object with a microarray for immobilizing biomolecules that has been produced by the aforementioned method according to the invention.

It is a further object of the present invention to specify a use of the microarray both for immobilizing biomolecules and for analyzing biomolecules present in a sample.

In some embodiments, the present invention achieves the above-mentioned objects by the use of a microarray according to the invention for immobilizing biomolecules in an immobilization zone, the biomolecules being optionally: nucleic acids, in particular deoxyribonucleic acids or ribonucleic acids; peptides; or proteins, in particular enzymes or antibodies.

In some embodiments, the present invention achieves the above-mentioned objects by the use of a microarray according to the invention for analyzing biomolecules present in a sample, the biomolecules being optionally: nucleic acids, in particular deoxyribonucleic acids or ribonucleic acids; peptides; or proteins, in particular enzymes or antibodies.

There are, then, various ways of advantageously configuring and developing the teaching of the present invention. In relation to this, reference is made to the text herein as well as the following explanation of exemplary embodiments of the invention with reference to the drawings. In conjunction with the explanation of the exemplary embodiments of the invention with reference to the drawings, a general explanation of exemplary configurations and developments of the teaching will also be provided.

With respect to further advantageous configurations of the microarray according to the invention for immobilizing biomolecules, of the method according to the invention for producing a microarray for immobilizing biomolecules, of the microarray for immobilizing biomolecules produced by the method, and of the uses of the microarrays according to the invention, reference is made to the description and to the accompanying examples to avoid repetition.

### Example 1

Comparative analysis of nitrocellulose coating. Atomic force microscopy images were recorded in intermittent contact mode in air at room temperature at a speed of 1 Hz. Roughness was determined for the entire image at a resolution of 256×256 pixels.

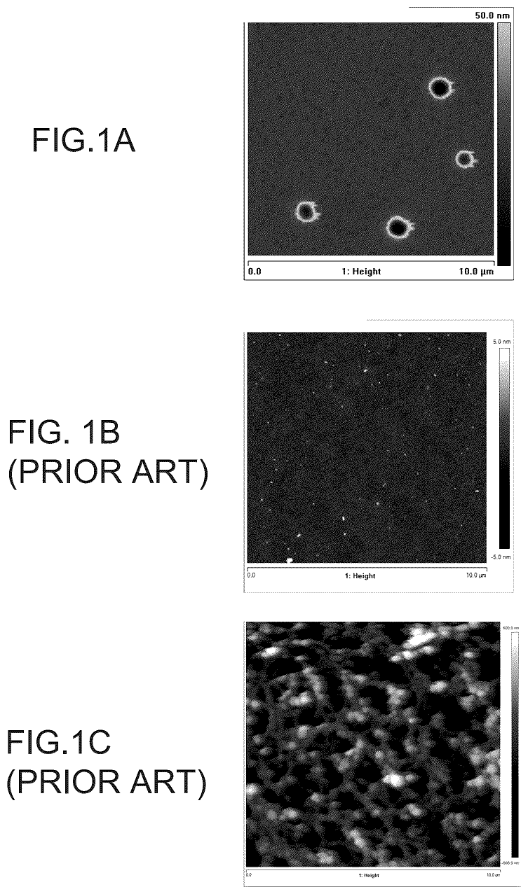

FIG. 1A illustrates the advantageous structure of the surface of the coating of a microarray according to the invention with a layer thickness of 72 nm compared to the smooth surface of a comparably thin nitrocellulose coating of the known microarray “PATH® Protein Microarray Slide” with a layer thickness of 30 nm, as shown in FIG. 1B, and to the surface quality of a 3-dimensionally structured porous nitrocellulose coating in the micrometre range (layer thickness of 12.5 μm) of the known microarray “ONCYTE® SuperNOVA”, as shown in FIG. 1C.

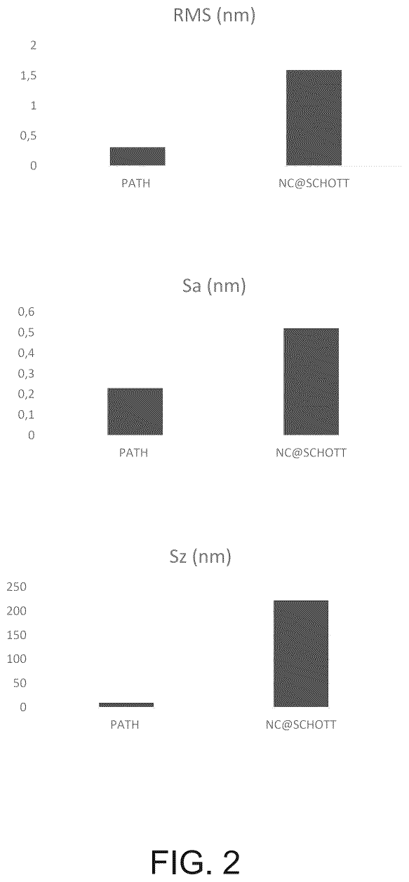

FIG. 2 shows the advantageously adjusted roughness parameters RMS, Sₐ and S_{z} of a microarray according to the invention compared to the known “PATH® Protein Microarray Slide”. The roughness values were ascertained from atomic force microscopy images having an edge length of 30 μm×30 μm. The roughness of the surface of the “ONCYTE® SuperNOVA” microarray is not shown. However, as can already be seen by looking at FIG. 1C, the roughness of the surface of the “ONCYTE® SuperNOVA” microarray is very high. In fact, the ascertainable value for RMS roughness is over 80 nm.

### Example 2

Comparative transmittance properties. Transmittance measurement was carried out by spectrophotometry. For this purpose, the sample was placed in the beam path perpendicular to the optical axis. Since the sample is coated on both sides, the beam passes through both layers. The transmittance values therefore apply to the entire sample; for one layer, they are expected to be even higher.

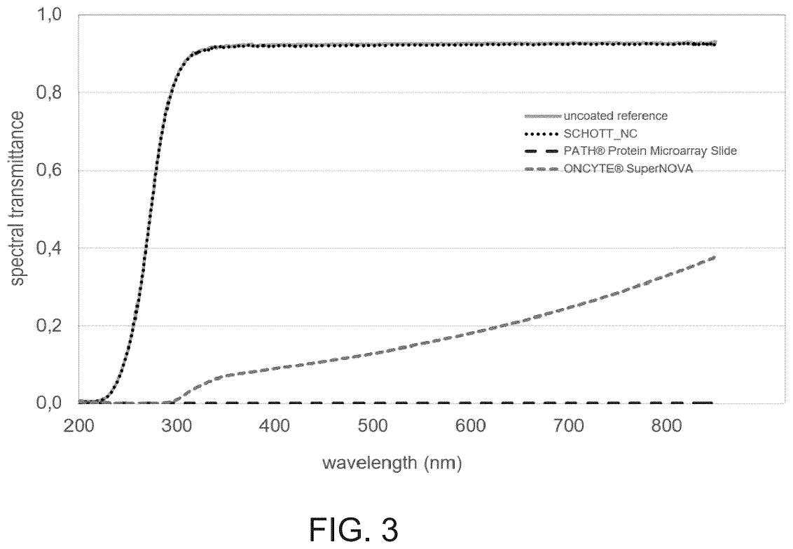

The transmittance profile of the microarray according to the invention is shown in FIG. 3. Advantageously, the transmittance of the SCHOTT_NC microarray coated with nitrocellulose according to the invention is virtually identical to that of the uncoated reference substrate. PATH® and ONCYTE® have a distinctly lower transmittance.

### Example 3

Measurement of background intensity versus nitrocellulose layer thickness. To determine layer thickness, the layer was partially removed mechanically and the layer thickness measured at three different points using a white light interferometer. The value specified is the mean and standard deviation of the measurement points over at least 5 samples prepared from different batches. The associated background intensity was determined according to Example 4.

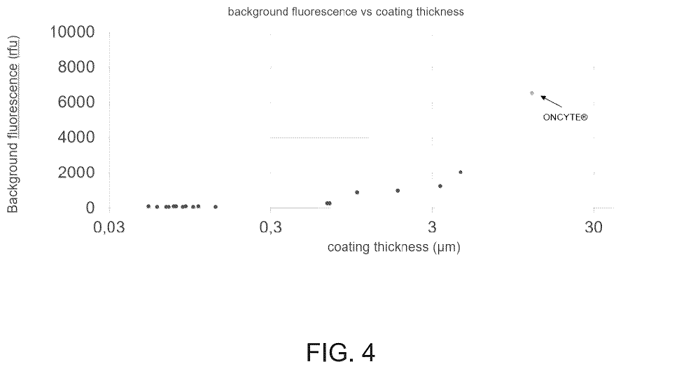

The background fluorescence of nitrocellulose-coated microarrays rises—as shown in FIG. 4—with layer thickness. For comparison, the background fluorescence of ONCYTE®, which has a distinctly higher background fluorescence, is shown.

### Example 4

Comparative measurement of background intensity. By a fluorescence scanner at an excitation wavelength of 532 nm and pixel size of 10 μm, background intensity was ascertained over the entire sample, leaving out an edge region of 5 mm. The level of the intensity measured can be adjusted by varying the gain and is kept constant for comparison of different samples.

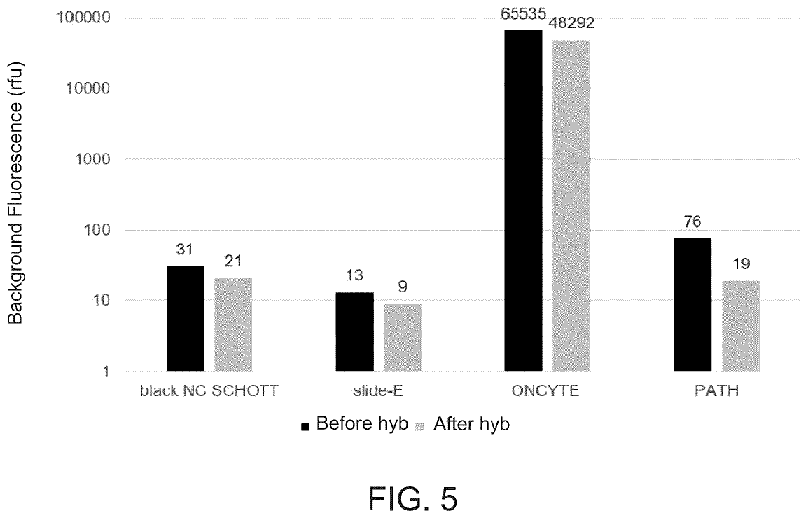

FIG. 5 shows the background fluorescence of the SCHOTT_NC coating compared to SCHOTT Slide-E and the nitrocellulose-coated microarrays ONCYTE® and PATH® from Grace Biolabs, before and after a hybridization (hyb) reaction in each case. The microarrays were scanned at a gain of 150 and a wavelength of 532 nm. The scale chosen for the background fluorescence is in logarithmic units.

### Example 5

Comparative spot morphology. To assess spot morphology, spatially resolved surface emission was depicted in 2D and 3D surface plots. What is advantageous here is a homogeneous distribution of intensity over the entire spot.

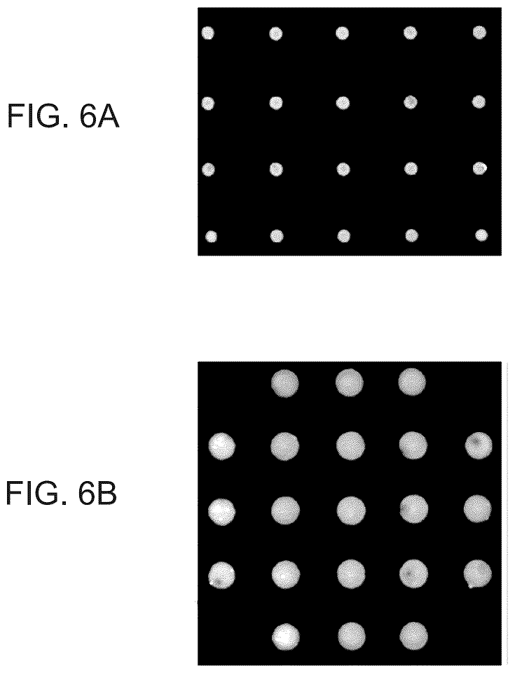

FIGS. 6A-6B illustrate the advantageous spot morphology of 500 picolitre spots in an array on the thin nitrocellulose coating of a SCHOTT_NC microarray according to the invention with a spot diameter of approx. 150 μm (FIG. 6A)) compared to the spot diameter of approx. 200 μm on a 3 μm thick nitrocellulose coating (FIG. 6B)).

### Example 6

Comparative determination of dynamic range. Within the dynamic range, the intensities of the spots depend on the protein solution used. Outside the dynamic range, saturation of intensity is apparent.

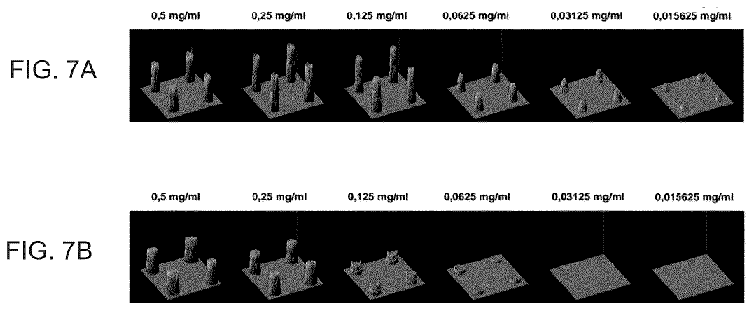

FIGS. 7A and 7B show 3D surface plots of the spots applied to a microarray according to the invention having a thin nitrocellulose coating with a root mean square (RMS) roughness of at least 0.5 nm (SCHOTT_NC, FIG. 7A)) versus protein concentration (rabbit anti-goat IgG) for determining the dynamic range. As can be seen, the dynamic range of the microarray according to the invention is distinctly extended compared to a functionalized 2-dimensional epoxy array (FIG. 7B).

Lastly, it should be expressly noted that the above description of exemplary embodiments of the microarray according to the invention for immobilizing biomolecules, of the method according to the invention for producing a microarray for immobilizing biomolecules, of the microarray for immobilizing biomolecules produced by the method, and of the uses of the microarrays according to the invention serves only for discussion of the claimed teaching, but does not restrict said teaching to the exemplary embodiments.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A microarray for immobilizing biomolecules, comprising:
a glass or glass-ceramic substrate; and
a nitrocellulose coating disposed at least regionally on a first planar surface of the substrate, the nitrocellulose coating configured to serve as an immobilization zone for biomolecules, a layer thickness of the nitrocellulose coating being between 10 and 150 nm, the nitrocellulose coating being optically clear, and the nitrocellulose coating having a root mean square (RMS) roughness of at least 0.5 nm.

2. The microarray of claim 1, wherein the layer thickness of the nitrocellulose coating is between 10 and 100 nm.

3. The microarray of claim 1, wherein the nitrocellulose coating has a root mean square (RMS) roughness of between 0.5 and 2 nm.

4. The microarray of claim 1, wherein the nitrocellulose coating has a roughness in terms of a mean of absolute deviations (Sₐ) of between 0.3 and 1 nm.

5. The microarray of claim 1, wherein the nitrocellulose coating has a roughness in terms of a peak-to-valley height (S_{z}) of between 150 and 300 nm.

6. The microarray of claim 1, wherein the microarray has a transmittance in a wavelength range between 300 and 800 nm of at least 85%.

7. The microarray of claim 1, wherein:
the layer thickness of the nitrocellulose coating is between 45 and 75 nm;
the nitrocellulose coating has a root mean square (RMS) roughness of between 1.45 and 1.65 nm;
the nitrocellulose coating has a roughness in terms of a mean of absolute deviations (Sₐ) of between 0.5 and 0.6 nm;
the nitrocellulose coating has a roughness in terms of a peak-to-valley height (S_{z}) of between 210 and 230 nm; and
the microarray has a transmittance in a wavelength range between 300 and 800 nm of between 90% and 95%.

8. The microarray of claim 1, wherein the first planar surface of the substrate is functionalized and comprises at least one of the following:
functional ester groups;
functional ether groups;
functional epoxy groups;
functional aldehyde groups;
functional free carboxyl groups; or
functional free amino groups.

9. The microarray of claim 8, wherein the first planar surface of the substrate is functionalized by an adhesion promoter layer which comprises at least one of the following:
functional ester groups;
functional ether groups;
functional epoxy groups;
functional aldehyde groups;
functional free carboxyl groups; or
functional free amino groups.

10. The microarray of claim 1, wherein an intrinsic fluorescence of the microarray is less than 100 relative fluorescence units at an excitation wavelength of 532 nm and a gain of 150.

11. The microarray of claim 1, further comprising biomolecules immobilized in the immobilization zone, wherein the biomolecules comprise at least one of: nucleic acids, deoxyribonucleic acids, ribonucleic acids, peptides, proteins, enzymes, or antibodies.

12. The microarray of claim 1, characterized in that a spotting volume of 500 pl applied in the immobilization zone has a spot diameter of between 130 and 170 μm.

13. The microarray of claim 1, wherein the microarray is stable when stored at between 4° C. and 40° C. for more than 2 months.

14. A method for producing a microarray, comprising:
providing a coating solution, the coating solution comprising between 0.2% and 3.0% by weight of highly pure nitrocellulose in an organic solvent, between 0.5% and 10.0% by weight of (3-glycidyloxypropyl)trimethoxysilane (GPTS), and between 0.3% and 20.0% by weight of water;
applying the coating solution to a glass or glass-ceramic substrate by dipping the substrate into the coating solution and then withdrawing it from the coating solution; and
depositing a nitrocellulose coating from the coating solution on a first planar surface of the substrate by drying, the nitrocellulose coating having a layer thickness of between 10 and 150 nm, wherein the deposited nitrocellulose coating is optically clear and has a root mean square (RMS) roughness of at least 0.5 nm.

15. The method of claim 14, wherein the coating solution additionally comprises between 0.01% and 3.0% by weight of biotin or between 0.01% and 3.0% by weight of streptavidin.

16. The method of claim 14, wherein the coating solution comprises:
between 0.5% and 1.5% by weight of highly pure nitrocellulose in an organic solvent;
between 2.0% and 4.0% by weight of GPTS; and
between 0.3% and 15.0% by weight of water.

17. The method of claim 16, wherein the coating solution comprises:
between 0.8% and 1.2% by weight of highly pure nitrocellulose in an organic solvent;
between 2.8% and 3.2% by weight of GPTS; and
between 4.8% and 5.2% by weight of water.

18. The method of claim 17, wherein the coating solution comprises:
1% by weight of highly pure nitrocellulose in an organic solvent;
3% by weight of GPTS; and
5% by weight of water.

19. The method of claim 14, wherein a drawing speed for dipping and subsequent withdrawal is between 5 and 15 cm per minute at a temperature of the coating solution of between 18° C. and 25° C.

20. The method of claim 14, wherein the drying takes place over a period of at least 5 minutes at a temperature between 18° C. and 90° C.

21. The method of claim 14, further comprising functionalizing at least the first planar surface of the substrate, the functionalizing taking place during the applying of the coating solution or taking place before the applying of the coating solution in a separate step by applying an adhesion promoter layer.

22. The method of claim 14, wherein the organic solvent is amyl acetate, isopropanol, 1-propanol, tetrahydrofuran, or dimethyl sulfoxide.
